(19) United States
(12) Patent Application Publication
(10) Pub. No.: US 2009/0131927 A1
(43) Pub. Date: May 21, 2009

(54) METHOD AND APPARATUS FOR REMOTE DETECTION OF RF ABLATION

(76) Inventors: Nathan Kastelein, St Louis, MO (US); Ashwini K. Pandey, Marlborough, MA (US); Yi-Ren Woo, Livermore, CA (US); Raju R. Viswanathan, St. Louis, MO (US); Gareth T. Munger, St Louis, MO (US); Christopher D. Minar, New Prague, MN (US); Roger G. Riedel, JR., Mahtomedi, MN (US)

Correspondence Address: HARNESS, DICKEY, & PIERCE, P.L.C, 7700 Bonhomme, Suite 400, ST. LOUIS, MO, 63105, US

(21) Appl. No.: 12/272,185

(22) Filed: Nov. 17, 2008

Related U.S. Application Data
(60) Provisional application No. 60/989,445, filed on Nov. 20, 2007.

(51) Int. Cl. A61B 18/18; A61B 18/14

(52) U.S. Cl. 606/33; 606/41

(57) ABSTRACT

Devices for the generation and detection of an ablative plasma discharge in a subject are presented. Methods of use, including navigation and operation of the devices to facilitate minimally invasive therapeutic procedures are disclosed.

### CROSS-REFERENCE TO RELATED APPLICATIONS

This application claims the benefit of U.S. Provisional Patent Application Ser. No. 60/989,445, filed Nov. 20, 2007. The disclosure of the above-referenced application is incorporated herein by reference.

### FIELD OF THE INVENTION

This invention relates to the detection of the progress of RF ablation in a medical procedure by non-invasive means.

### BACKGROUND

Minimally invasive intervention systems include navigation systems, such as the Niobe™ magnetic navigation system developed by Stereotaxis, St. Louis, Mo. Such systems typically comprise an imaging means for real-time guidance and monitoring of the intervention; additional feedback is provided by a three-dimensional (3D) localization system that allows real time determination of the catheter or interventional device tip position and orientation with respect to the operating room and, through co-registered imaging, with respect to the patient.

RF devices are used in the medical field to create openings through blocked passages, or to otherwise remove unwanted material. During the process of removal, the RF device in many cases generates a plasma within a local region near its tip. Examples of such devices include guidewires or catheters with electrodes at the tip for delivery of RF energy. When such devices are used for ablative material removal, a small region of plasma is created at the device tip which both heats and dissociates a small layer of material in the tissue. This usually requires a sufficient concentration of ions in the vicinity of the device electrode. As the device is pushed into the tissue, the opening thus created in the tissue is enlarged. In some instances where there may be an insufficient ion concentration, a current passes through the device electrode and into the tissue without the generation of a plasma. In this latter case, the electrode and the local tissue simply heats up, without ablative removal of material or the creation of a passage in the tissue, and this could lead to overheating of the device electrode and/or the local tissue.

During the course of a medical procedure using such an RF device, it is desirable to avoid such overheating and to know whether or not ablative material removal with a local plasma discharge is actually occurring. While the device is inserted interventionally into the patient and usually imaged with fluoroscopy, there is no method available at present to determine this.

The present invention addresses this need and provides for a method and apparatus for the detection of a plasma discharge during RF ablation.

### SUMMARY

Generally this invention relates to RF devices such as catheters, guidewires, endoscopes, and the like. One preferred embodiment is a Radio Frequency guidewire. In this preferred embodiment, the guidewire could be magnetically enabled for remote magnetic navigation, while in another it could be manually operated. The wire is preferably made of electrically conductive material with an insulating jacket, and has an exposed electrode portion at its distal end. In practice, the wire is inserted through a blood vessel to a partially or totally occluded portion of the vessel, with the distal tip placed just proximal to the occlusion. As RF energy is delivered through the wire, with the right ionic concentration in the region surrounding the distal tip, a plasma discharge and ablative material removal occurs in the vicinity of the electrode. This can be a continuous process if the tip is advanced into the occluded lesion, resulting in the opening of a passage.

The plasma discharge occurs as a dielectric breakdown due to locally high electric fields in the vicinity of the electrode tip. As such, it is accompanied by a burst of fluctuating electric fields over a range of frequency values as the molecular dissociation occurs. This burst can be detected as noise by a suitable pickup antenna, or with a device such as an AM radio receiver. The detection efficiency of the noise signal can be enhanced by suitable hardware. The detected signal can be processed and displayed in a variety of ways, or simply directly conveyed to the user as an audio signal with audio speakers. The processing can look for specific signatures such as frequency content or time course of the signal or intensity profile.

As non-limiting examples, the visual display of the signal can show intensity over a range of frequencies, a simple processed indication of on or off, or the presence of certain pre-selected frequencies. The wire could be controlled by a remote navigation system such as a magnetic navigation system or mechanically driven navigation system. The visual display or indication of plasma discharge could be shown on an X-ray image monitor (one focus of attention in a catheterization laboratory), or on the user interface display of a remote navigation system, or both. Audio speakers to render the information as an audible sound can be provided in the procedure room, or in a remote navigation system control room, or both.

The long body of the wire itself can act as an antenna that picks up the signal at its distal end in the form of a weak electric current. The detection apparatus or antenna can thus be placed at or near the proximal portion of the device. In one embodiment, the proximal portion of the wire can itself be looped to enable better inductive coupling between the detection antenna and the wire body. The detection antenna can be connected to electronic amplification circuitry to further enhance the detected signal.

The display of this information to the user can aid the user in determining whether the wire placement is appropriate for ablation; if it is not, as determined from the displayed ablation information, the user can reposition the wire, infuse saline, or otherwise change the configuration of the wire or modify its distal environment until a successful ablation is indicated. At this point, the wire can be pushed onward, or steered or deflected suitably in order to open a passageway through the occlusive lesion.

The continuous availability of real-time ablation information can greatly help the process of navigating through a lesion.

### BRIEF DESCRIPTION OF THE DRAWINGS

FIG. 1 is a schematic diagram showing an RI ablation device used within a patient, together with a pickup antenna and an amplification, processing and display system for displaying plasma discharge information;

FIG. 2 is a schematic diagram of an RF wire looping spool and a pickup coil for detection of plasma discharge radio noise. Illustrating one possible spooling method for forming a loop in the proximal portion of the wire, that can aid in better detection efficiency due to inductive enhancements;

FIG. 3 is a schematic diagram of an RF wire looping spool and a pickup coil embedded in a flexible drape or patch for detection of plasma discharge radio noise; and

FIG. 4 is a schematic flowchart depicting a workflow for an ablative RF procedure employing the present invention.

Corresponding reference numerals indicate corresponding parts throughout the several views of the drawings.

### DETAILED DESCRIPTION OF THE INVENTION

According to the preferred embodiment of the present invention, an RF-capable medical device (such as a catheter, guidewire, or endoscope) is navigated and positioned within a patient's anatomy just proximal to the desired ablation region. The navigation process could be manual, or in the case where a remote navigation system is used it can be used to correspondingly actuate the medical device and navigate it to the desired location. The device is connected to an RF generator that drives RF power through the device tip electrode into the region to be ablated; the generator is controlled by the physician performing the procedure. As described herein, the RF-capable medical device here is taken to be a RF guidewire, but it could be any RF-capable navigable device. In one preferred embodiment, the generator could be automatically driven from a remote navigation system when its correct location is confirmed either automatically from image or other sensed data, or manually.

In ablative RF power delivery mode, the RF power delivery results in a localized plasma discharge at the electrode dissociating molecules within a localized region around the electrode when the local ionic concentration is sufficiently high. Such a discharge lasts only for a very brief time interval, and therefore ablatively dissociates material around the electrode without leading to persistently high temperatures in the region. If the local conductivity properties are not suitable for ablative power delivery, predominantly resistive power delivery occurs, which could lead to significant temperature increases. The ablative mode of power delivery is therefore the preferable mode of operation for the RF power delivery system in procedures where material removal (such as occlusion removal) is desired, as for instance is the case in coronary or peripheral vessel lesion treatment.

Embodiments of the present invention detect the occurrence of such a plasma discharge. The ablative RF power delivery results in fluctuating electric fields near the device tip electrode leading to a burst of electromagnetic noise in a fairly wide band. For example, the inventors have found that RF power delivery at a frequency of about 450 KHz can lead to a burst of electromagnetic noise in a frequency range of about 450 KHz-1 MHz. This noise can be detected as radio static, for example with an AM radio receiver, in one preferred embodiment. Alternatively, a specialized receiver coil can be used to pick up the electromagnetic noise, the signal passed through an amplifier (optionally with a tuned circuit), and then conveyed to speakers for an audible signal of the plasma discharge or visually displayed on a suitable monitor. In a preferred embodiment, the RF device itself can be used as an antenna that picks up the noise signal at the distal end and propagates it to the proximal portion of the device. The corresponding current or voltage fluctuations at the proximal portion can be detected with a receiver coil inductively coupled to the RF device. In another preferred embodiment, the signal from the RE device can be shunted to other circuitry within the RF generator (where the proximal end is connected), and the signal suitably amplified and conveyed to the user.

When the noise signal is detected through inductive coupling of a receiver coil, in one preferred embodiment the RF device is itself looped in the form of a coil with at least approximately one turn over its proximal portion. This results in a corresponding noise magnetic field through the loop, which can be detected by a receiver coil with better detection efficiency. In some cases where the length of wire within the subject is shielded by the subject's body due its dielectric properties, this better detection efficiency can result in better signal amplification.

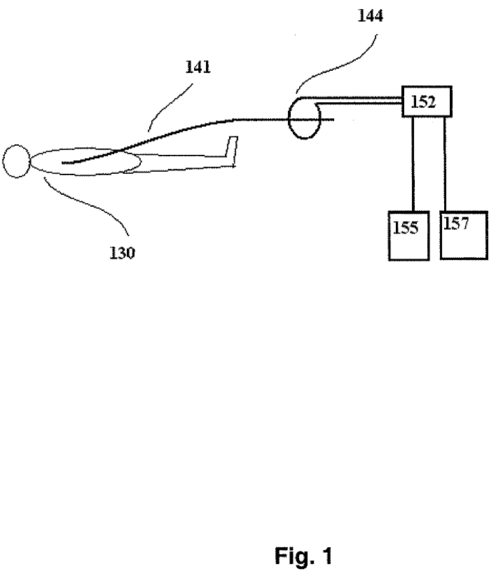

FIG. 1 is an illustration of one embodiment of the plasma noise signal detection system in accordance with the present invention. For purposes of specific example, a RF wire 141 is shown inserted into patient 130. A pickup or detection coil 144 is placed near the proximal portion of the wire; the coil is connected to electronic circuitry 152 that includes amplification circuitry and possibly tuning circuitry as well, tuned to cover a band of frequencies. While the figure shows the pickup coil placed close to a proximal portion of the wire, in one preferred embodiment it can also be placed at some distance from it, such as 20 cm or more.

In one preferred embodiment the signal pickup coil and electronics can be integrated in a single device, for example a standard AM radio or a specialized radio electronics device. Alternatively the electronics can be a separate electronics box, or it could be incorporated as part of signal processing circuitry (possibly as part of a specialized computer card). In the latter case, the signal can be analyzed for frequency content and to identify a characteristic signature of the plasma discharge radio noise. Such a signature could comprise, for example, one or more of: range of frequencies present, presence of signal within the major portion of a pre-defined band of frequencies, distribution of intensity profile over a pre-defined range of frequencies, peaks in intensity over specific sub-bands in a pre-defined band of frequencies, or absence or low signal over specific sub-bands in a pre-defined band of frequencies. These examples of specific signature are provided for purposes of non-limiting example only, and other suitable or convenient signatures could be defined by those skilled in the art.

The signal, either with or without processing, is then conveyed to a set of audio speakers 155, or alternatively or additionally to a visual display 157 where the signal is suitably displayed visually. The visual display can simply be an indication of the presence of plasma discharge noise, or it can be more detailed information derived from the above examples of specific signature. In a preferred embodiment where a remotely navigated RF medical device is used, the visual display can be shown on a user interface monitor that is part of the remote navigation system. Examples of such remote navigation system modalities are magnetic navigation, mechanically actuated interventional navigation systems that use motor-controlled pull-wires, electrostrictive actuation methods, hydraulic actuation, or magnetostrictive actuation. Whether or not a remote navigation system is used, the visual display can in another preferred embodiment be shown on a fluoroscopy system monitor where the device is visualized within the subject. In still another preferred embodiment, the visual display can be shown both on a remote navigation system user interface and on a fluoroscopy monitor.

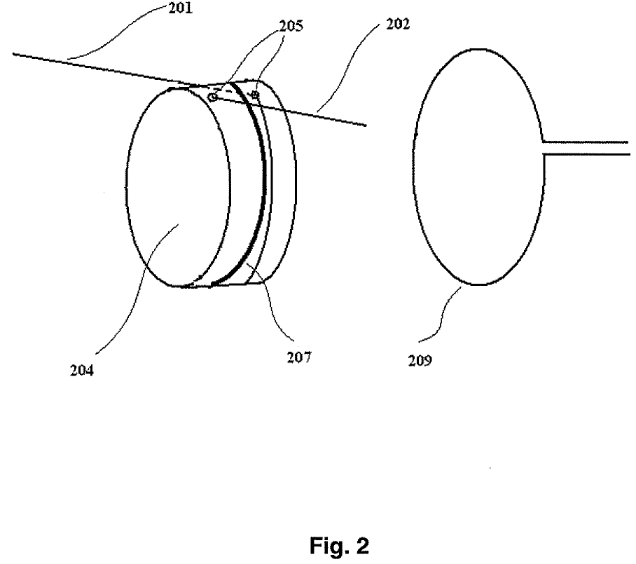

FIG. 2 is an illustration of a guidewire spooling device used together with a signal pickup coil in order to improve signal detection efficiency. The RF guidewire is spooled through a spool 204 with suitable spooling holes 205 and preferably including a helical groove and a corresponding helical ridge portion 207 that permits easy and rapid spooling of the wire. The distal and proximal portions of the wire extend out from portions 201 and 202 of the wire, respectively. A signal pickup coil 209 is placed anywhere within a range of distances from the spooled wire. Inductive coupling between the spooled wire loop and the pickup coil results in enhanced pickup even in some cases where direct detection of the radio signal from the distal portion of the device may be partially shielded by the subject's body mass. An example of a range of distances over which such inductive coupling can enhance the signal can be anywhere from 1 mm to 10 meters, for purposes of non-limiting example only.

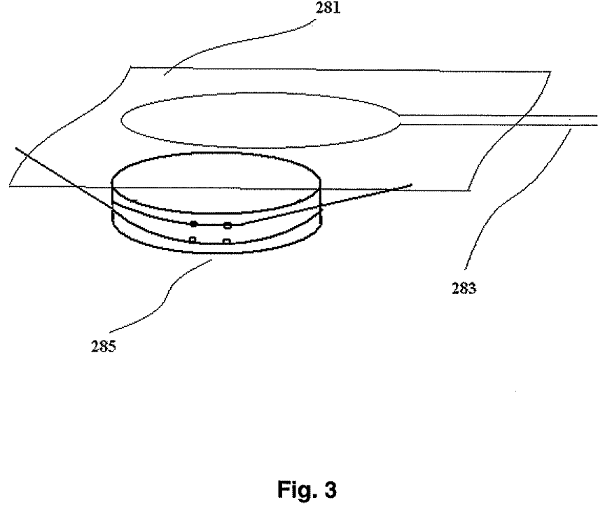

FIG. 3 shows a signal pickup coil embedded in a flexible thin sheet in the form of a surgical drape or patch 281, placed close to or on top of a spool 285 for spooling the guidewire. For example, during an interventional medical procedure the spool can be placed at a convenient location on the patient table or directly on the patient. In such a procedural setting, the drape or patch can be easily laid across the spool to yield good inductive coupling. The leads 283 of the pickup coil are connected to signal amplification electronics (not shown), as before.

### OPERATION

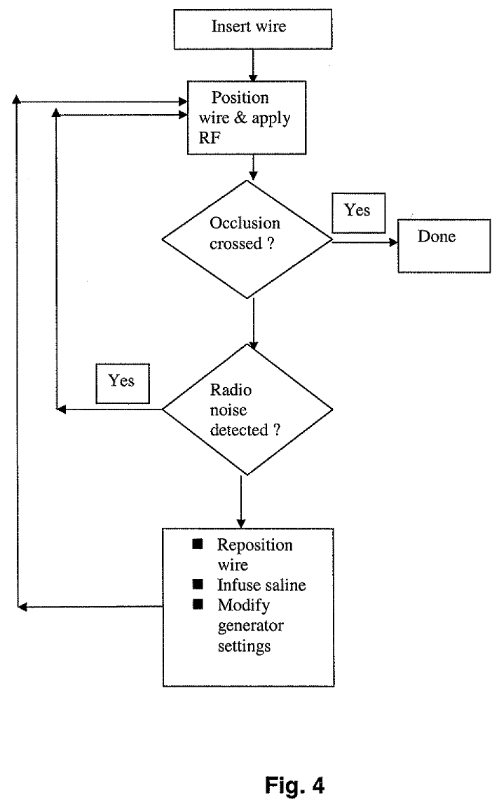

FIG. 4 shows a high-level flowchart and procedural workflow for crossing an occluded vessel according to the preferred embodiment of the method of the present invention. The wire is inserted into the patient and guided to and positioned at the occlusion lesion suitably. RF power is applied and the plasma radio noise signal detection of the present invention used to detect the presence of a plasma discharge, indicating ablative material removal. If the radio noise is detected, the wire is suitably positioned/advanced and RF power is applied again. If radio noise is not detected, one or more of the following are performed: repositioning of the wire, infusion of saline to the occlusion site to enhance local ionic concentration, or modification of the RF generator power delivery settings, followed again by application of RF power. The process is continued until the occlusion is crossed.

While the specific medical device in described has been a RF guidewire, it should be apparent that any other suitable medical device can also be used for RF power delivery. Likewise, the method of wire navigation can be manual, or it can employ a remote navigation system, such system being actuated through magnetic, mechanical, electrostrictive, magnetostrictive or hydraulic actuation means. In such cases the medical device is suitably configured to permit corresponding actuation. Other such generalizations will be apparent to those skilled in the art and the scope of the invention is only limited by the attached claims.

The detection of ablation also facilitates the automation of the process in conjunction with a remote navigation system. For example the device can be positioned and RF energy applied until ablation occurs. Once ablation has been detected, the remote navigation system can reposition the device, and RF energy again applied. If unsuccessful ablation occurs, the system can automatically reposition the device for a more successful ablation, or adjust other parameters, such as injecting saline at the ablation site, or adjusting the parameters of the RF generator.

Furthermore, the system can be provided with a library or stored data about the RF signature of successful and unsuccessful ablations, and with other problems, or the system can store current procedure information about the RF signature of successful and unsuccessful ablations. The signals generated can be compared with the library data or the current procedure information, and the visual and audible information can be adjusted so that more information about the nature and character of the ablation is provided to the physician.

The methods and apparatus of the various embodiments of this invention allow the physician to be more certain when ablation has or has not occurred, and thus perform the procedure faster and more efficiently, either manually or with automated assistance.

What is claimed is:

1. An RF medical device for ablation of material in a subject, the device comprising:
an elongated medical device to transmit RF energy through a passageway in the subject's body, the elongated medical device comprising a distal end for application of RF ablative energy;
an RF generator capable of generating plasma discharges in the neighborhood of the RF elongated device distal end; and
an external RF signal detection means for detecting RF signals corresponding to successful RF ablation.

2. The medical device of claim 1, wherein the external RF signal detection means further comprises signal processing means.

3. The medical device of claim 1, further comprising a user interface comprising at least one of an image display, an audio speaker, a visual signal, a haptic indicator.

4. The medical device of claim 1, wherein the external RF signal detection means further comprises an AM radio.

5. The medical device of claim 1, wherein the external ur signal detection means further comprises a dedicated signal pick-up coil.

6. The medical device of claim 1, wherein the elongated medical device is further coiled around a wire spool adjacent its proximal end.

7. The medical device of claim 1, wherein the external ur signal detection means further comprises signal amplification electronics.

8. The medical device of claim 1, wherein at least part of the external RF signal detection means is embedded in a flexible drape for positioning near the subject.

9. The medical device of claim 1, wherein the external RF signal detection means further comprises signal processing and analysis means for the detection of radio signal signatures.

10. The medical device of claim 9, further comprising means for display of the radio signal signatures.

11. A method for the detection of the ablation of material in a subject, the method comprising:
navigating an elongated medical device to transmit RF energy through a passageway in the subject body;
operating an RF generator, the generator being connected to the elongated medical device and capable of generating plasma discharges in the neighborhood of the RF elongated device distal end; and
detecting an RF signal associated with the plasma discharges generated in the neighborhood of the elongated medical device distal end.

12. The method of claim 11, further comprising processing the detected RF signal;

13. The method of claim 12, wherein processing the detected RF signal comprises amplifying the detected signal.

14. The method of claim 12, wherein processing the detected RF signal comprises analyzing the detected signal for the existence of specific signal signatures.

15. The method of claim 11, further comprising communicating with a medical device user through a user interface means.

16. The method of claim 11, further comprising generating an audio signal in response to the detection of an RF signal.

17. The method of claim 11, wherein the step of detecting an RF signal further comprises detecting a signal generated in a dedicated pickup coil.

18. A method of performing a minimally invasive therapy in a lumen of a subject, the method comprising:
navigating an RF-enabled elongated medical device to the proximity of a subject lumen occlusion, the RF-enabled elongated medical device being connected to an RF generator and capable of generating plasma discharges in the neighborhood of its distal end;
applying RF energy through the elongated medical device;
detecting a signal through an external detection device, and determining the presence of a plasma related signal;
adjusting the RF generator parameters and therapy parameters to improve the likelihood of generating a plasma discharge in the neighborhood of the elongated medical device distal end;
evaluating the progress of the therapy; and
iterating through steps i) to v) to enable further therapy progress.

19. The method of performing a minimally invasive therapy in a subject according to claim 18, wherein the step of adjusting the RF generator parameters and therapy parameters comprise adjusting RF power settings, injecting saline, and adjusting RF frequency settings.

20. The method of performing a minimally invasive therapy in a subject according to claim 18, wherein the step of evaluating an RF signal through an external detection device further comprises processing the signal and interfacing with the user through user interface means.

21. The method of claim 18, where navigating the elongated medical device is performed with a remote navigation system.

22. The method of claim 21, where the remote navigation system is a magnetic navigation system.

23. The method of claim 21, where the remote navigation system is a mechanically actuated navigation system.
